(19) United States
(12) Patent Application Publication
(10) Pub. No.: US 2022/0183875 A1
(43) Pub. Date: Jun. 16, 2022

(54) DISPOSABLE PENILE ERECTION DEVICE

(71) Applicant: Peter L. Levy, San Francisco, CA (US)

(72) Inventor: Peter L. Levy, San Francisco, CA (US)

(21) Appl. No.: 17/086,394

(22) Filed: Oct. 31, 2020

(51) Int. Cl. A61F 5/41

(52) U.S. Cl. CPC A61F 5/41; A61F 2005/415; A61F 2005/411

(57) ABSTRACT

A non-surgical, non-drug, non-waiting period penile disposable erection device having a urethra insertion rod structure with rod assembly inflating character providing rigidity to a flaccid penis.

### BACKGROUND

### Field of the Invention

The present invention relates generally to male erectile dysfunction and more specifically, the invention relates to a non-surgical, non-drug, non-waiting period penile erection solution.

### Background of the Invention

The inability to develop or maintain an erection of the penis during sexual activity is called Erectile Dysfunction (ED) aka impotence. Treatment involves In many cases, a trial of pharmacological therapy with a PDE5 inhibitor such as sildenafil. In some cases, treatment can involve inserting prostaglandin into the urethra, injecting smooth muscle relaxants and vasodilators into the penis, a penile implant, a penis pump, or vascular reconstructive surgery. Erectile dysfunction is the most common sexual problem in men.

Medications

The PDE5 inhibitors sildeniafil, tadalifil and vardenafil are prescription drugs which are taken orally. Additionally, a cream has been approved in Canada as a first line treatment for ED. Penile injections can involve medications: papaverine, phentolamine, and prostaglandin El aka alprostadil

In addition to injections, there is an alprostadil suppository that can be inserted into the urethra. Once inserted, an erection can begin within 10 minutes and last up to an hour. However, all medications to treat ED may have side effects.

Penile Prosthesis

A penile prosthesis is another treatment option for men with ED. These devices are either malleable, bendable or inflatable. The simplest type of prosthesis consists of a pair of malleable rods surgically implanted within the erection chambers of the penis. With this type of implant the penis is always semi-rigid and merely needs to be lifted or adjusted into the erect position to initiate sex.

A penile implant is usually used when there is a clear medical cause for ED and when the problem is unlikely to resolve or improve naturally or with other medical treatments. Sometimes a penile prosthesis is implanted during surgery to reconstruct the penis when scarring has caused erections to curve.

The Workings of Penile Prosthetics

The inflatable penile prosthesis consists of two attached cylinders—a reservoir and a pump—which are placed surgically in the body. The two cylinders are inserted in the penis and connected by tubing to a separate reservoir of saline. The reservoir is implanted under the rectus muscles in the lower abdomen. A pump is also connected to the system and sits under the loose skin of the scrotal sac, between the testicles.

To inflate the prosthesis, a patient presses on the pump. The pump transfers saline from the reservoir to the cylinders in the penis, inflating them and causing an erection. Pressing on a deflation valve at the base of the pump returns the fluid to the reservoir, deflating the penis and returning it to the normal flaccid state.

The bottom line is placement of penile implants requires surgery and they include. possible risks, complications and follow-up care.

Most men consider penile implants if they aren't a candidate for other treatments or can't get an erection sufficient for sexual activity by using other methods. Penile implants can also be used to treat severe cases of a condition that causes scarring inside the penis, leading to curved, painful erections, Peyronie's disease.

Risks of Penile Implant Surgery

Risks include Infection. As with any surgery, infection is possible. You might be at an increased risk of infection if you have a spinal cord injury or diabetes; Implant problems. New penile implant designs are reliable, but in rare cases the implants might malfunction. Surgery is necessary to remove, repair or replace a broken implant; Internal erosion or adhesion. In some cases, an implant might stick to the skin inside the penis or wear away the skin from inside the penis. Rarely, an implant breaks through the skin. These problems are sometimes linked to an infection.

Treating an Implant Infection

Infections after penile implant surgery typically occur in the first few weeks or possibly years later. Early infections can cause swelling of the scrotum, pus buildup and fever. Later infections might involve persistent or recurrent long-term pain. Surgery to remove the implant is likely necessary to treat an infection. Replacing a penile implant can be complicated and can lead to a buildup of scar tissue and a decrease in penis length.

Much can go wrong with the surgical approach, before, during and after the procedure. Some are: The implant has more parts that could malfunction than does any other implant; Requires a reservoir inside the abdomen; Is mechanically more complicated than is a semi rigid implant; Provides less firm erections than does a three-piece implant; Results in a penis that is always slightly rigid; Puts constant pressure on the inside of the penis, which can cause injury; Can be difficult to conceal under clothing; Before penile implant surgery you might also need to temporarily stop taking aspirin and anti-inflammatory drugs, which can increase your risk of bleeding.

Patients may be given IV antibiotics to help prevent infection. They will likely need to take medications to ease pain. Mild pain might persist for several weeks. Patients will likely need to return to the doctor to have their stitches removed. Penile implants are the most invasive and least often chosen treatment for erectile dysfunction. The 10-year device survival is between 60 and 80 percent.

In addition, although an alternative, there is no perfect prosthetic penis as they present problems of their own. What is needed are alternative solutions to ED.

Current methods to treat ED fail for any number of reasons. The various drugs are less than 100 percent effective and many have undesirable side effects such as blurry vision, headaches etc. Most drugs have a waiting period before they are effective. The vacuum pump has numerous problems such as the danger of using constriction devices to trap blood flow and numerous unpleasant side effects

What is needed are a quick, temporary, non-chemical or drug, minimum of or tolerable temporal physical side-effects, non-surgical solutions to ED, the largest single sexual problem inflicting men. What is needed is a solution not dependent on drugs or requires injections or vacuum devices all of which have numerous problems. What is needed are solutions without a waiting period before the solution becomes effective.

### SUMMARY

The present invention discloses a non-surgical, non-drug, non-waiting period penile erection solution. A disposable penile erection device is disclosed which has a soft cap with a surface fitting conformably to a penis glans surface and having an opening more-or-less in the cap center aligned with a glans surface meatus. A flexible rod with one end coupled more-or-less perpendicular to the cap has rod length between 3 to 14 inches and unexpanded diameter of between 1/16 to ½ inch and a base terminating distal end base to guide and support a rod-membrane expansion assembly. The membrane is flexible stretchable more-or-less cylindrical and firmly coupled to the cap under hermetical seal, with membrane extending axially and radially surrounding and enclosing the rod. The device inserted into a penis urethra and expanded provides structural stiffness and penile erection.

### BRIEF DESCRIPTION OF DRAWINGS

Specific embodiments of the invention will be described in detail with reference to the following figures.

FIG. 1 illustrates an anatomical broad view of a disposable penile erection device in an embodiment of the invention,

FIG. 2 shows a detail cross-section view of a deposable penile erection device in an embodiment of the invention.

FIG. 3 displays a fluidic expandable urethra insertion assembly for a disposable penile erection device in an embodiment of the invention

FIG. 4 shows an inflated anchored membrane component for a disposable penile erection device in an embodiment of the invention.

FIG. 5 illustrates and array of thin staves bound with circumferential hoops and base mechanical spring assembly enclosed in an expandable membrane for a disposable penile erection device in an embodiment of the invention

### DETAILED DESCRIPTION

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

### Objects and Advantages

An object of the invention is to provide a non-surgical penile erection solution.

Another object of the invention is to provide a non-drug penile erection solution.

Yet another objective of the invention is to a non-waiting period penile erection solution.

Another object of the invention is to provide a quickly installable penile erection device

Yet another object of the invention is to provide a disposable penile erection device.

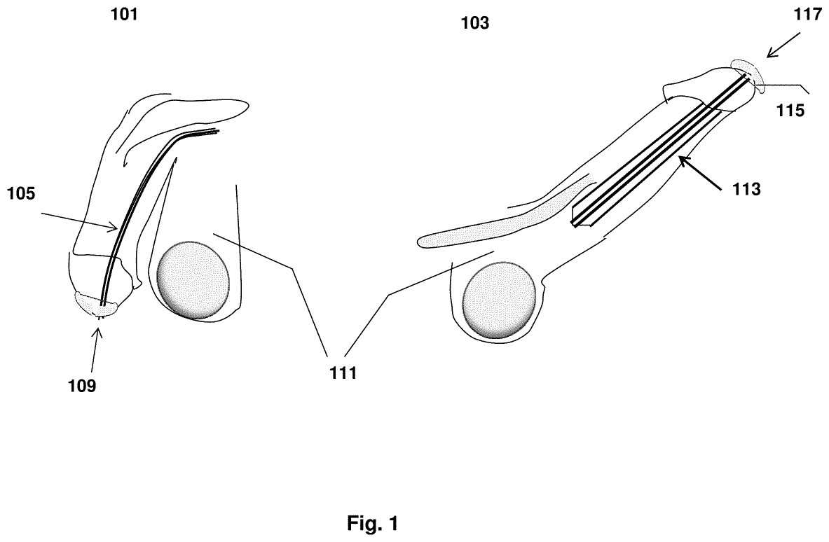

FIG. 1 illustrates an anatomical broad view of a disposable penile erection device in an embodiment of the invention,

A flaccid penis 101 is shown with a disposable penile erection device 105 inserted into a urethra through the meatus and is coupled to a elastic cap 109 conformably adjacent to glans. An erect penis 103 is shown with an installed disposable penile erection device 113 extending from the cap 115, fitted conformably adjacent to the glans 117, and extending toward the penis root and scrotum 111 or radix, longitudinally along and inside of the urethra.

In an embodiment of the invention a soft cap 109 115 with a surface fitting conformably to a penis glans surface and having an opening 117 more-or-less in the cap center aligned with a glans surface meatus. a flexible rod assembly will have one end coupled more-or-less perpendicular to the cap 109 115, with a rod length between 3 to 14 inches and unexpanded diameter of between 1/16 to ½ inch and having a base terminating distal end. A flexible stretchable more-or-less cylindrical membrane is firmly coupled to the cap 109 115 under hermetical seal, with membrane extending axially and radially surrounding and enclosing the rod.

The inserted portion of a erection device assembly 105 113 has an expansion mechanism that may be fluidic or mechanical in structure. Upon expansion from the erection device internals, the urethra is stretched in girth adding structural stiffness to a penis much like blood flow to the Corpus cavernosum will accomplish upon penis erection. Any natural tendency for blood flow to the Corpus cavernosum is not impeded and may add to attain structural stiffness and support erection.

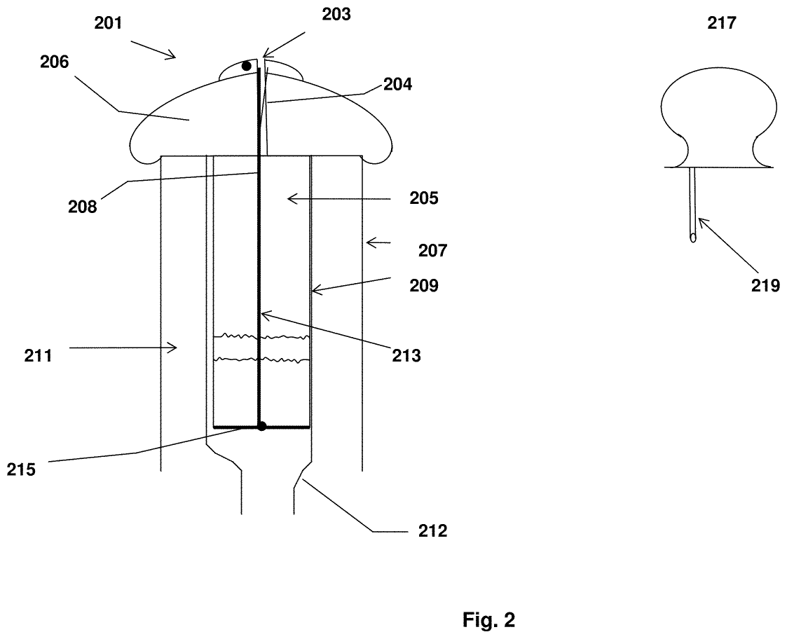

FIG. 2 shows a detail cross-section view of a deposable penile erection device in an embodiment of the invention. A disposable penile erection cap 201 fits conformably adjacent to the penis glans 206 and is concentrically located over the penis meatus. The cap 201 has a coupled rod 208 more or less perpendicularly anchored to the cap 201 which is inserted through the meatus opening and into a urethra 212 such that the cap 201 is flush with the penis glans 206 when a flexible rod 208 component is fully inserted. The rod 208 has two ends, the first end is rigidly coupled the cap 201 and the rod 208 distal end is flexibly coupled to the rod terminal membrane anchor 215 and provides structural support for device insertion. The membrane wall 209 is wrapped and surrounds the rod 208 and is of elastic flexible expandable material which is air/fluid sealed to provide a closed and sealed expandable volume upon entry.

The cap 201 has a central opening 203 for a air needle valve lock 204, which is used in conjunction with a complementary hand pump 217 having a needle valve 219 for inflating or expanding a flexible expandable membrane wall 209, the membrane top edge is coupled to the cap 201 and extends the full length of and encloses the rod 208 for a fluid sealed insertion into a urethra. Air 205 or fluid 211 from the hand pump 271 will inflate and expend the membrane 209 laterally against the penis and urethra inside wall 207 and longitudinally from pressure on a rod bottom membrane floor 215. The membrane wall 209 expansion and extension will expand the urethra 212 and provide rigidity and erection to the penis 207.

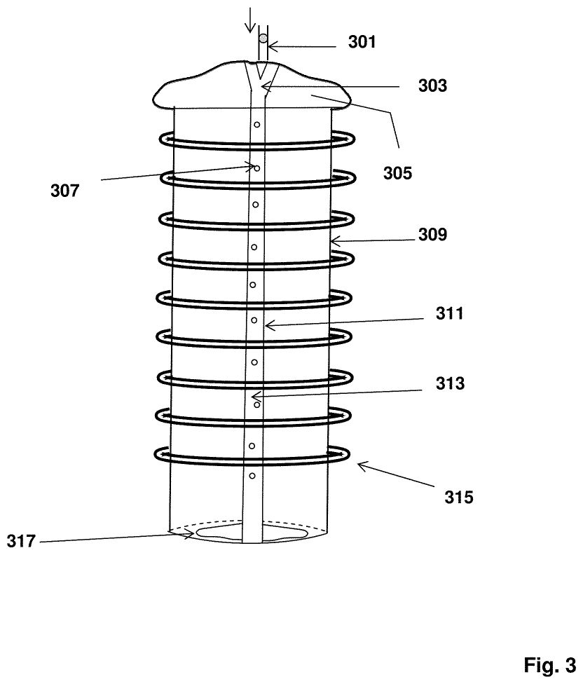

FIG. 3 displays a fluidic expandable urethra insertion assembly for a disposable penile erection device in an embodiment of the invention.

A mechanism and assembly for an air/fluidic expandable urethra insertion penile erection device is centered around a thin hollow flexible rod component 307, coupled more-or-less perpendicular to a glans conforming soft cap 305. The flexible rod 307 cap 305 has a simple needle valve air/fluid lock 303 for an external needle valve 301, shown inserted into a cap lock 303 which is contiguously and hermetically coupled to the rod 307 component. The rod 307 component acts as a insertion guide and has at least one perforation or side opening so incoming air/fluid into the rod fills the expandable elongated membrane 309 which encloses and hermetically seals the rod 307 and is hermetically seal coupled to the cap 305. The membrane 309 may have a slightly thicker contiguous bottom 317 terminal end, such that air/fluid pumped 301 into rod 307 via an open lock valve 303 flows into the membrane 309 chamber and the expands radial outward and axially downward toward the rod distal end and 317 coupled to the membrane 309 terminal end. Air/fluid pumped 301 into the cap-valve assembly 305 via an external needle valve 301 pump are removed or taken offline. A penile erection device deployment requires membrane 309 fluid deflation and rod-cap assembly 305 removal for disposal after use. The membrane 307 is made from elastic-rubber-synthetic material fluid holding and expandable/stretchable and in some embodiments with rings 315 on membrane 309 surface which increase friction stabilizing forces.

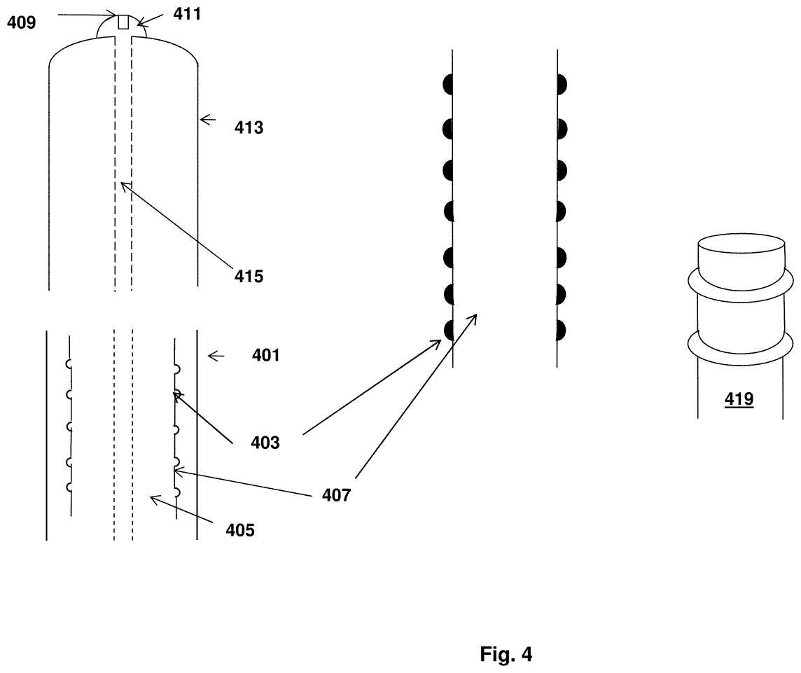

FIG. 4 shows an expanded and friction anchored membrane component of a penile erection device in an embodiment of the invention.

A device assembly is shown with airlock 409 cap 411 coupled to a rod-cap subassembly 415 and expandable membrane 407 at the cap 411 and extending axially and radially with internal air/fluid 405 pressure on the membrane 407 and against a urethra 401 413. The membrane 407 may have surface ridges 403 or spirals which are round or triangular in cross-section or more ring-like 419 protrusions 419 for anchoring the penile erection device membrane 407 into the urethra 413 401 interior wall. The membrane wall 407 will be made from a set of elastic stretchable material consisting of latex, rubber, nitrile, lamb cecum, polyurethane and hermetic non-porous synthetic material. The membrane wall 407 can come applied with a set of non-prescription lubricants including relaxant creams, gels, rubs, topical analgesics, counterirritants menthol, methylsalicylate, camphor, salicylates, capsaicin and CBD family relaxants. Membrane lubricants with prescription can be used where not prohibited.

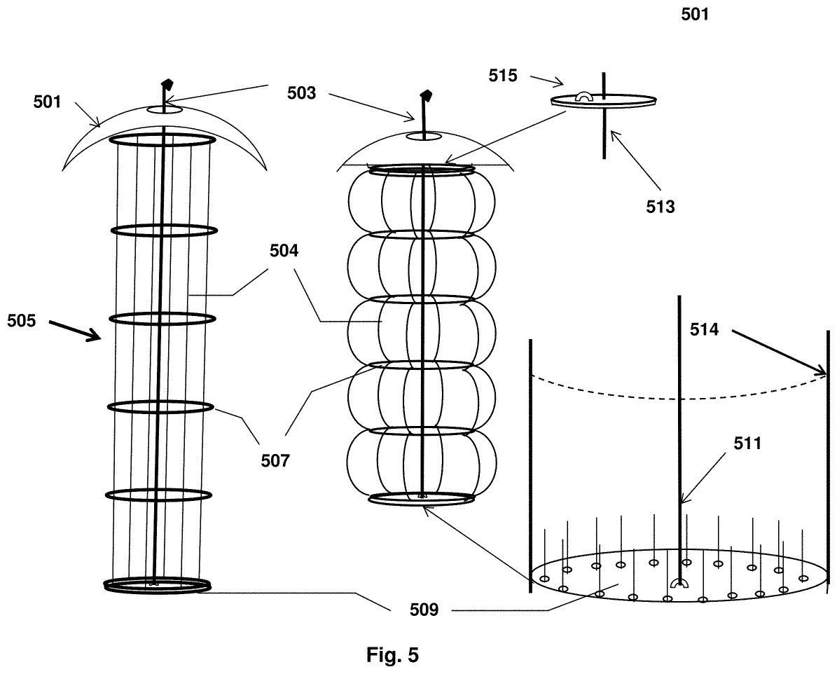

FIG. 5 illustrates and array of thin staves bound with circumferential hoops and base mechanical spring assembly for a penile erection device in an embodiment of the invention

An array of uniformly spaced staves 504 coupled to and extending from a cap 501 to the edges of an anchor more-or-less circular plate base, the length of the staves 504 coupled to and extending from the cap 501 to the circular base plate 509. The cap-staves-base plate form a cylindrical rib cage 505, with staves 504 as ribs with hoops 507 at uniform separation lengths along the stave lengths, with the cap 501 and base plate 509 acting as head hoops for the spring fiber rib cage 505. The staves 504 are thin spring bendable plastic or fiber which bows outward from a circular array center upon rib cage 505 contraction. A base plate is coupled string 511 513 or fiber coupled from the base plate 509 center to a cleat 515 or snap fastener in the cap 501 assembly, serves to expand a membrane 514 516 encased rib cage 505 laterally upon string 511 513 length contraction when manipulated from the cap 501 end and secured by cleat 515 or fastener. The rib cage fibers 504 may be made of plastic, composite, nylon, synthetic fiber or other bendable semi-rigid material, such that limited compression will bow the fiber without breaking, to accomplish a spring bowing lateral force on a membrane 516 against a urethra inner wall.

Therefore, while the invention has been described with respect to a limited number of embodiments, those skilled in the art, having benefit of this invention, will appreciate that other embodiments can be devised which do not depart from the scope of the invention as disclosed herein. Other aspects of the invention will be apparent from the following description and the appended claims.

What is claimed is:

1. A disposable penile erection device comprising:
a soft cap with a surface configured to fit conformably to a penis top and having an opening substantially in the cap center, configured to align with the urethra the cap center configured to align with a urethra;
a flexible rod with one end coupled substantially perpendicular to a cap, a length of the rod being between 3 to 14 inches and an unexpanded diameter of between 1/16 to ½ inch and having a base terminating distal end; and
a flexible stretchable cylindrical membrane firmly coupled to the soft cap under hermetical seal, with the cylindrical membrane extending axially and radially surrounding and enclosing the rod, and
whereby the penile erection device is configured to provide structural stiffness to a penis member from fluidic expansion of the cylindrical membrane from within the urethra.

2. A disposable penile erection device as in claim 1 wherein the rod base distal end is flexibly coupled to the cylindrical membrane terminal end anchor.

3. A disposable penile erection device as in claim 1 further comprising the tubular flexible rod with at least one tube side opening permitting fluid to flow from the cap opening into the membrane via the tubular rod.

4. A disposable penile erection device as in claim 1 further comprising the soft cap with an air/fluid needle valve orifice.

5. A disposable penile erection device as in claim 1 further comprising the flexible rod as an array bundle of semi-flexible staves uniformly spaced and constrained at the cap and a rod's base plate, and having non-stretchable hoops at uniform segments along the rod length to provide spring bowing of the staves upon a string length contraction for radial expansion.

6. A disposable penile erection device as in claim 5 further comprising the soft cap with a cleat or fastener for a string anchored at the flexible rod distal base.

7. A disposable penile erection device as in claim 1 further comprising a flexible stretchable cylindrical membrane with an outside surface ring or spiral ridges which are round or triangular in cross-section protrusions for providing anchoring friction.

8. A disposable penile erection device as in claim 1 further comprising a cylindrical membrane material from a set of elastic stretchable material consisting of latex, rubber, nitrile, lamb cecum, polyurethane and hermetic non-porous synthetic material.

9. A disposable penile erection device as in claim 1 further comprising a cylindrical membrane applied with a set of non-prescription lubricants including relaxant creams, gels, rubs, topical analgesics, counterirritants menthol, methylsalicylate, camphor, salicylates, capsaicin and CBD family relaxants.

10. A method for a disposable penile erection device further comprising the steps of:
providing a soft cap with a surface fitting conformably to a penis top and having an opening substantially in the cap aligned with a urethra;
coupling a flexible rod with one end substantially perpendicular to the soft cap, a length of the rod being between 3 to 14 inches and an unexpanded diameter of between 1/16 to ½ inch and having a base terminating distal end; and
coupling a flexible stretchable substantially cylindrical membrane firmly to the soft cap under hermetical seal, with the cylindrical membrane expandable axially and radially surrounding and enclosing the flexible rod,
whereby the disposable penile erection device provides structural stiffness to a penis member.

11. A method for a disposable penile erection device as in claim 10 further comprising the steps of coupling a rod base terminating distal end flexibly to a cylindrical membrane.

12. A method for a disposable penile erection device as in claim 10 further comprising the steps of providing a slender tubular rod with at least one side opening channeling fluid flow from the cap orifice filling or draining the cylindrical membrane volume via the at least one tubular rod side opening.

13. A method for a disposable penile erection device as in claim 10 further comprising the steps of providing the soft cap with an air/fluid needle valve cap orifice.

14. A method for a disposable penile erection device as in claim 10 further comprising the steps of providing the flexible rod as an array bundle of semi-flexible staves uniformly spaced and constrained at the cap and the flexible rod base ends, and having non-stretchable hoops at uniform segments along the rod length to provide spring bowing of the staves upon rod array bundle contraction providing cylindrical membrane radial expansion.

15. A method for a disposable penile erection device as in claim 10 further comprising the step of having the soft cap with a cleat or fastener for a string anchored at the flexible rod distal end base which can be distended at the cap and secured.

16. A method for a disposable penile erection device as in claim 10 further comprising the steps of providing the cylindrical membrane with an outside surface ring or spiral ridge protrusions of round, semi-round, triangular or other polygonal cross-section protrusions providing anchoring friction to the cylindrical membrane.

17. A method for a disposable penile erection device as in claim 10 further comprising the step of using the cylindrical membrane material from a set of elastic stretchable material consisting of latex, rubber, nitrile, lamb cecum, polyurethane and hermetic non-porous synthetic material.

18. A method for a disposable penile erection device as in claim 10 further comprising the step of using the cylindrical membrane from a set of non-prescription lubricants including relaxant creams, gels, rubs, topical analgesics, counterirritants menthol, methylsalicylate, camphor, salicylates, capsaicin and CBD family relaxants.
